(19) United States
(12) Patent Application Publication
(10) Pub. No.: US 2016/0045527 A1
(43) Pub. Date: Feb. 18, 2016

(54) TREATMENT OF SLEEP APNEA WITH A COMBINATION OF A CARBONIC ANHYDRASE INHIBITOR AND AN ALDOSTERONE ANTAGONIST

(71) Applicant: VIVUS, INC., Mountain View, CA (US)

(72) Inventors: Charles H. Bowden, Mountain View, CA (US); Craig Peterson, Mountain View, CA (US)

(21) Appl. No.: 14/825,284

(22) Filed: Aug. 13, 2015

Related U.S. Application Data
(60) Provisional application No. 62/037,244, filed on Aug. 14, 2014.

(51) Int. Cl. A61K 31/7048; A61K 31/433; A61K 31/137; A61K 31/585; A61K 31/575; A61K 9/00; A61K 31/423

(52) U.S. Cl. CPC A61K 31/7048; A61K 9/0053; A61K 31/433; A61K 31/423; A61K 31/585; A61K 31/575; A61K 31/137

(57) ABSTRACT

This invention relates generally to methods and pharmaceutical formulations useful in treating patients suffering from sleep apnea, including obstructive sleep apnea syndrome (OSAS). Treatment is effected by administering a carbonic anhydrase inhibitor to the patient in combination with an aldosterone antagonist. Formulations containing a therapeutically effective amount of a carbonic anhydrase inhibitor and a therapeutically effective amount of an aldosterone antagonist are provided as well.

### RELATED APPLICATIONS

This application claims the benefit of provisional application U.S. Ser. No. 62/037,244, filed Aug. 14, 2014, the contents which are herein incorporated by reference in their entirety.

### TECHNICAL FIELD

The invention relates generally to the treatment of sleep apnea, and more particularly relates to the treatment of sleep apnea with a combination of a carbonic anhydrase inhibitor and at least one additional active agent that includes an aldosterone antagonist. The invention finds utility in the fields of medicine and pharmacotherapy.

### BACKGROUND

Apnea occurs when an individual breathes very shallowly or stops breathing completely over a time period of 10 seconds or more, resulting in a drop in blood oxygen level. Apneas usually occur during sleep and cause the individual to wake or transition from a deep level of sleep to a more shallow sleep state. “Hypopneas” refer to decreases in breathing that also result in hypoxemia, but are less severe than apneas. Generally, apnea refers to a reduction in airflow or chest wall movement to less than 25% of baseline, while hypopnea refers to a reduction in airflow or chest wall movement to a level in the range of about 25% to about 70% of baseline. See K. Banno et al. (2007) Sleep Medicine 8 (4):400-426. Hypopnea has also been defined as a decrease in ventilation of at least 50% and a consequent reduction in arterial saturation of 4% or more. G. C. Mbata et al. (2012) Ann. Med. Health Sci. Res. 2 (1):74-77.

The International Classification of Sleep Disorders—2^{nd} edition (ICDS-2) defines two categories of sleep-related breathing disorders, central sleep apnea syndrome (CSAS) and obstructive sleep apnea syndrome (OSAS). Mixed sleep apneas involve both CSAS and OSAS. The distinction between CSAS and OSAS relates to the mechanism that causes the respiratory disturbance. CSAS involves a dysfunction in ventilatory control in the central nervous system (CNS), with a reduction in impulses transmitted from the CNS to the respiratory muscles. OSAS, which is much more common than CSAS, is a disorder that is caused by physical obstruction of the upper airway. The obstruction typically results from abnormal control of the muscles that maintain the patency of the upper airway, and/or abnormal craniofacial anatomy. Common risk factors for OSAS include obesity, enlarged tonsils and adenoids, and cranofacial abnormalities.

OSAS has emerged as a common sleep disorder that is associated with excessive daytime sleepiness as well as more significant problems. The altered physiology induced by the periodic hypoxia and reoxygenation that characteristically occur in OSA result in oxidative stress, endothelial dysfunction, sympathetic activation, and activation of the inflammatory cascade, al of which favor cardiovascular disease and related comorbidities, including atherosclerosis, obesity, hypertension, heart failure, nocturnal cardiac arrhythmias, and an elevated risk of myocardial infarction and stroke. See, e.g., Sleep Apnea: Implications in Cardiovascular and Cerebrovascular Disease, 2^{nd} Ed., Bradley et al., eds. (Informa Healthcare USA, Inc., 2010) (particularly Levitzky et al., Ch. 10, at p. 163; Friedman et al., Ch. 11, at p. 180; Lorenzo-Filho et al., Ch. 13, at p. 219; Siccoli et al., Ch. 14, at p. 237; Sorajja et al., ch. 15, at p. 261; and Yumino et al., ch. 17, at p. 302). A diagnosis of OSAS is typically made when repetitive apnea or hypopnea events occur during sleep, with 5-15 episodes/hour classified as mild OSAS, 15-30 episodes/hour classified as moderate OSAS, and over 30 episodes/hour classified as severe OSAS. Banno et al. (2007), citing Sleep-Related Breathing Disorders in Adults: Recommendations for Syndrome Definition and Measurement Techniques in Clinical Research, in Report of an American Academy of Sleep Medicine Task Force (1999), Sleep 22 (5):667-689.

The current standard of care for OSAS is the Continuous Positive Air Pressure (CPAP) technique, in which a continuous stream of compressed air is administered to the patient using a machine specifically designed for that purpose. CPAP, however, only provides benefits when used and does not consistently address the pathophysiology of OSAS. See Grote et al. (2000), Eur. Respir. J. 16:921-17. Other forms of treatment include intraoral mandibular advancement devices and craniofacial surgery. These methods are cumbersome and expensive, and although many pharmacological agents have been proposed and evaluated, no agent has proved to be successful in treating OSAS.

There is, therefore, a need for a simpler, straightforward method for treating an individual with OSAS; ideally, the method would also be effective in treating CSAS as well.

### SUMMARY OF THE INVENTION

The present invention addresses the aforementioned need in the art and provides a pharmacological treatment for patients suffering from sleep apnea. The treatment involves co-administration of a carbonic anhydrase inhibitor with an additional active agent or agents that include an aldosterone antagonist.

Any inhibitor of carbonic anhydrase can be used in the present methods and formulations. Without wishing to be bound by theory, applicants postulate that the effectiveness of including a carbonic anhydrase inhibitor in a combination therapy to treat sleep apnea results from the body's response to the metabolic acidosis caused by the drug (in contrast to the respiratory acidosis often experienced by sleep apnea sufferers, particularly OSAS sufferers). That is, as inhibition of carbonic anhydrase results in a decrease in blood pH—the reaction catalyzed by carbonic anhydrase is the reversible hydrolysis of carbon dioxide to give bicarbonate ion and a proton—the body works to compensate by breathing faster and deeper to expel excess carbon dioxide and thus restore equilibrium. Topiramate, zonisamide, and acetozolamide are representative of the many carbonic anhydrase inhibitors that may be used in the context of the present invention. Carbonic anhydrase inhibitors can also facilitate weight loss, which alleviates sleep apnea in many overweight and obese patients. In addition, sleep apnea tends to correlate with resistant hypertension, which in turn is associated with hyperaldosteronism. Aldosterone antagonists, it has been found, are useful in treating both sleep apnea and hypertension, and have a neutral or negative effect on weight. The combination of a carbonic anhydrase inhibitor with an aldosterone antagonist, as provided herein, is more effective than administration of either active agent alone. In addition, because the dosage of each active agent in the combination can be reduced relative to the dosages used in monotherapy, reduction in side effect profile is achieved as well.

In one aspect, then, the invention provides a method for treating sleep apnea in a patient by co-administering to the patient:

(a) a therapeutically effective amount of a carbonic anhydrase inhibitor; and

(b) a therapeutically effective amount of an aldosterone antagonist.

In another aspect of the invention, a method is provided for treating sleep apnea in a patient, comprising orally administering to the patient: a therapeutically effective amount of a carbonic anhydrase inhibitor selected from acetazolamide, brinzolamide, diclofenamide, dichlorphenamide, dorzolamide, furosemide, imidazole, methazolamide, phenylalanine, topiramate, and zonisamide; and a therapeutically effective amount of an aldosterone antagonist selected from spironolactone, canrenone, eplerenone, mexrenone, prorenone, and pharmaceutically acceptable basic addition salts thereof

In another aspect of the invention, a method is provided for treating sleep apnea in a patient, comprising orally administering to the patient, on a daily basis, a therapeutically effective amount of topiramate and a therapeutically effective amount of an aldosterone antagonist selected from spironolactone, eplerenone, and potassium canrenoate.

The invention additionally pertains to pharmaceutical formulations useful in the present methods of treating sleep apnea:

In one aspect of this embodiment, a pharmaceutical formulation for the treatment of sleep apnea is provided, the formulation comprising a therapeutically effective amount of a carbonic anhydrase inhibitor, and a therapeutically effective amount of an aldosterone antagonist.

In another aspect of this embodiment, a pharmaceutical formulation for the treatment of sleep apnea is provided, the formulation comprising a therapeutically effective amount of a carbonic anhydrase inhibitor selected from acetazolamide, brinzolamide, diclofenamide, dichlorphenamide, dorzolamide, furosemide, imidazole, methazolamide, phenylalanine, topiramate, and zonisamide; and a therapeutically effective amount of an aldosterone antagonist selected from spironolactone, canrenone, eplerenone, mexrenone, prorenone, and pharmaceutically acceptable basic addition salts thereof.

In a further aspect of this embodiment, a pharmaceutical formulation for the treatment of sleep apnea is provided, the formulation comprising a therapeutically effective amount of topiramate and a therapeutically effective amount of an aldosterone antagonist selected from spironolactone, eplerenone, and potassium canrenoate.

In another embodiment, the invention provides a packaged pharmaceutical preparation comprising: a carbonic anhydrase inhibitor and an aldosterone agonist; and instructions for administering, e.g., self-administering, the active agents in the treatment of sleep apnea. The active agents are present in amounts that are therapeutically effective for the treatment of sleep apnea, and may be in separate dosage forms or combined in a single dosage form, where the dosage forms are usually but not always orally administrable. The instructions for administration may include reference to an escalating dosing regimen wherein a lower daily dosage of one or more active agents is administered initially, with incremental increases at various designated time points thereafter. Ideally, a titration card is provided that sets forth the recommended dosages for at least four weeks.

### DETAILED DESCRIPTION OF THE INVENTION

It must be noted that, as used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the invention pertains. Specific terminology of particular importance to the description of the present invention is defined below.

The term “sleep apnea,” as the term is used herein, is intended to include both OSAS and CSAS.

“Treatment of OSAS” refers to treating obstructive sleep apnea syndrome as defined earlier herein, but does not exclude the possibility that the individual being treated may also have some degree of CSAS.

In addition, by “treating sleep apnea” applicants are referring to (1) the elimination of nighttime apneas and/or hypopneas, (2) a reduction in the number of apneas and/or hypopneas per hour and/or per night, and/or (3) the amelioration of the extent of each apnea and/or hypopnea event experienced by the individual undergoing treatment (as may be determined, for instance, by an increase in airflow or in the amplitude of chest wall movement). While some methods of treatment and pharmaceutical formulations herein may also alleviate excessive daytime sleepiness, any such effect is incidental to the present method, which treats sleep apnea by virtue of effecting (1), (2), and/or (3) as explained above.

When referring to an active agent, applicants intend the term “active agent” to encompass not only the specified molecular entity but also its pharmaceutically acceptable, pharmacologically active analogs, including, but not limited to, salts, esters, amides, prodrugs, conjugates, active metabolites, crystalline forms (including polymorphs), enantiomers, and other such derivatives, analogs, and related compounds.

By the terms “effective amount” and “therapeutically effective amount” of a compound is meant a nontoxic but sufficient amount of an active agent to provide the desired effect, i.e., treatment of sleep apnea as manifested by the elimination of nighttime apneas and/or hypopneas, a reduction in the number of apneas and/or hypopneas per hour and/or per night, and/or amelioration of the extent of each apnea and/or hypopnea event experienced by the individual undergoing treatment.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient, provides a measurable time delay before drug is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

The invention involves administration of a carbonic anhydrase inhibitor and an aldosterone antagonist to a patient suffering from sleep apnea. Carbonic anhydrase inhibitors are generally imidazoles (such as imidazole per se), imidazole derivatives, sulfamates (such as topiramate), and sulfonamides (such as zonisamide). Any carbonic anhydrase inhibitor may be advantageously employed in conjunction with the present invention. Examples of suitable carbonic anhydrase inhibitors include, without limitation, acetazolamide (Diamox™), brinzolamide, diclofenamide, dichlorphenamide (Daranide™), dorzolamide, furosemide, imidazole, methazolamide (Neptazane™), phenylalanine, topiramate, and zonisamide. Carbonic anhydrase inhibitors also include selective inhibitors of the cyclooxygenase-2 enzyme (“cox 2 inhibitors”), such as such as celecoxib, valdecoxib, rofecoxib, etoricoxib, and the like. Preferred carbonic anhydrase inhibitors for use in conjunction with the present invention include, without limitation, acetazolamide, brinzolamide, diclofenamide, dichlorphenamide, dorzolamide, furosemide, imidazole, methazolamide, phenylalanine, topiramate, zonisamide, celecoxib, valdecoxib, rofecoxib, and etoricoxib, with acetazolamide, zonisamide, and topiramate particularly preferred. The daily dose of topiramate effective to treat sleep apnea according to the method of the invention, when administered orally, is generally in the range of about 15 mg to about 800 mg, more typically in the range of about 25 mg to about 400 mg, preferably in the range of about 45 mg to about 250 mg, and optimally in the range of about 45 mg to about 100 mg. Examples of specific suitable doses include, without limitation, 15 mg, 20 mg, 23 mg, 30 mg, 40 mg, 46 mg, 50 mg, 60 mg, 69 mg, 75 mg, 80 mg, 85 mg, 92 mg, 100 mg, 150 mg, and 200 mg. The daily dose may be undivided, such that the carbonic anhydrase inhibitor is administered once a day, or the daily dose may be divided into two to four individual doses. Preferably, the topiramate is administered in sustained release form, as will be discussed infra, either once or twice daily to achieve a daily dosage in the aforementioned ranges. It will be appreciated that the daily dose of topiramate as well as other carbonic anhydrase inhibitors normally represents on the order of 10% to 200%, more generally 15% to 100%, and most typically 25% to 100%, of the daily dose known and/or prescribed for previously known indication(s) (as set forth, for example, in the Physicians' Desk Reference), using the same mode of administration.

In a preferred embodiment, the dosage of the carbonic anhydrase inhibitor is increased gradually at the outset of therapy, generally over a period of about three to ten weeks, more usually over a period of about three to about eight weeks, starting with a relatively low initial dose, in order to reduce the likelihood of undesirable side effects. With topiramate, for example, a representative dosage regimen is as follows: administration of about 20 mg (e.g., 23 mg) daily for about the first 5-7 days of treatment; administration of about 45 mg (e.g., 46 mg) daily for the next 5-7 days; and optionally, administration of about 70 mg (e.g., 69 mg) daily for about the next 5-7 days followed by administration of about 90 mg (e.g., 92 mg) daily for the next 5-7 days; and, subsequently, ongoing administration of a daily maintenance dose in the ranges specified earlier herein.

The additional active agent is an aldosterone antagonist, i.e., an anti-mineralocorticoid compound that antagonizes the action of aldosterone at mineralocorticoid receptors. Any such compound may be used, although suitable compounds typically have the molecular structure of formula (I)

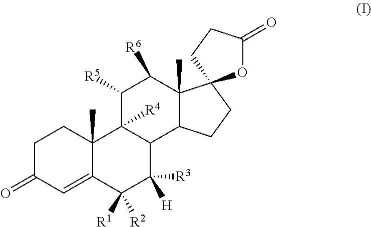

wherein:

(a) R¹ and R² are independently selected from H and OH, and R³ is selected from —COOR⁷ and —S(CO)R⁸ in which R⁷ and R⁸ are lower alkyl, or

R¹ is H and R² and R³ taken together form a double bond or a cyclopropyl ring;

(b) R⁴ and R⁵ are independently selected from H and OH, or R⁴ and R⁵ taken together form an epoxide ring; and

(c) R⁶ is selected from H and OH,

or are pharmaceutically acceptable basic addition salts of such compounds, in which the γ-butyrolactone ring at C-17 is opened and in anionic form, associated with a pharmaceutically acceptable cation. These basic addition salts, it will be appreciated, have the structure of formula (II):

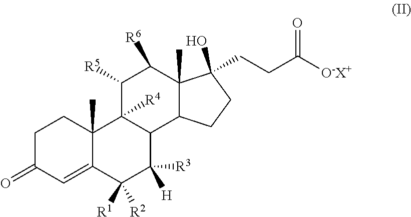

in which R¹ through R⁶ are defined as above with respect to structures of Formula (I), and X⁺ is a cation, e.g., an alkali metal cation such as a sodium or potassium ion, an alkaline earth metal cation such as a calcium ion, nitrogenous cations such as an ammonium ion or a quaternary ammonium ion, or the like. Unless indicated otherwise, compounds encompassed by the generic structure of Formula (I) include pharmaceutically acceptable basic addition salts as set forth in Formula (II).

In one preferred subgroup of these compounds, R¹ is H, R² and R³ taken together form a double bond, and R⁶ is H, such that the compound has the structure of Formula (III):

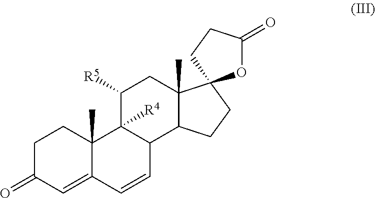

In another preferred subgroup, R¹, R², and R⁶ are H, such that the compound has the structure of Formula (IV):

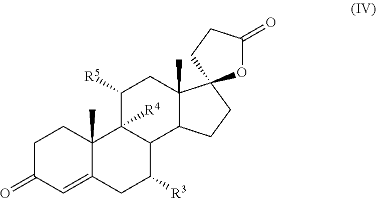

Preferred compounds encompassed by Formula (IV) include, without limitation, those wherein R³ is selected from —COOCH₃ and —SOCH₃.

Examples of such compounds include, without limitation, spironolactone (Formula (IV) where R³ is —SOCH₃ and R⁴ and R⁵ are H); mexrenone ((Formula (IV) wherein R³ is —COOCH₃ and R⁴ and R⁵ are H); eplerenone (Formula (IV) wherein R⁴ and R⁵ are taken together to form a 9,11-epoxide functionality and R³ is —COOCH₃); canrenone (Formula (III) wherein R⁴ and R⁵ are H); prorenone (Formula (I) wherein R² and R³ taken together form a cyclopropyl ring and R⁴ and R⁵ are H); and pharmaceutically acceptable basic addition salts of these compounds, having the structure of Formula (II).

The weight ratio of the daily dose of the carbonic anhydrase inhibitor to the daily dose of the aldosterone antagonist will depend on the particular active agents selected, but will generally be in the range of about 1:10 to about 2:1.

In a further embodiment, the carbonic anhydrase inhibitor and the aldosterone antagonist are co-administered with a therapeutically effective amount of a sympathomimetic amine.

Sympathomimetic amines, including the catecholamines, are amine drugs that mimic the actions of drugs that activate the sympathetic nervous system, such as epinephrine and norepinephrine. Sympathomimetic amines thus include amphetamine, benzphetamine, bupropion, chlorphentermine, colterol, diethylpropion, dopamine, dobutamine, ephedrine, epinephrine, epinine, ethylnorepinephrine, fenfluramine, fenoldapam, hydroxyamphetamine, ibopamine, isoetharine, isoproterenol, mephentermine, metaproterenol, metaraminol, methoxamine, methoxyphenamine, midodrine, norepinephrine, phendimetrazine, phenmetrazine, phentermine, phenylephrine, phenylethylamine, phenylpropanolamine, prenalterol, propylhexedrine, protokylol, ritodrine, terbutaline, tuaminoheptane, tyramine, and acid addition salts thereof, either organic or inorganic. Common acid addition salts of some of the aforementioned sympathomimetic amines include, without limitation, dobutamine hydrochloride, epinephrine bitartrate, ethylnorepinephrine hydrochloride, fenoldopam mesylate, hydroxyamphetamine hydrobromide, isoproterenol hydrochloride, mephentermine sulfate, metaraminol bitartrate, methoxamine hydrochloride, norepinephrine bitartrate, phentermine hydrochloride, phenylephrine hydrochloride, and terbutaline sulfate.

Preferably, the sympathomimetic amine is phentermine, bupropion, or chlorphentermine, with phentermine and bupropion particularly preferred. In an exemplary embodiment, the carbonic anhydrase inhibitor administered is topiramate, the aldosterone antagonist is spironolactone or eplerenone, and the sympathomimetic amine administered is phentermine, wherein the daily dose of topiramate is as given above, the daily dose of phentermine is in the range of about 2.5 mg to about 30 mg, preferably in the range of about 3.5 mg to about 17.5 mg, and the daily dose of the spironolactone or eplerenone is in the range of about 25 mg to about 200 mg, preferably in the range of about 25 mg to about 100 mg.

Administration of the active agents may be carried out using any appropriate mode of administration. Thus, administration can be, for example oral or parenteral, although oral administration is preferred.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical formulations and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). Oral administration and therefore oral dosage forms are generally preferred, and include tablets, capsules, caplets, solutions, suspensions and syrups, and may also comprise a plurality of granules, beads, powders, or pellets that may or may not be encapsulated. Preferred oral dosage forms are capsules and tablets.

As noted above, it is especially advantageous to formulate compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of each active agent, such that the unit dosage in each tablet or capsule is approximately 50% of the therapeutically effective amount.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are also preferred oral dosage forms, in which case the active agent-containing composition may be encapsulated in the form of a liquid or solid (the latter including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited earlier herein, which describes materials and methods for preparing encapsulated pharmaceuticals.

Oral dosage forms, whether tablets, capsules, caplets, or particulates, may, if desired, be formulated so as to provide for controlled release of the carbonic anhydrase inhibitor, the aldosterone antagonist, and/or the sympathomimetic amine, if present. In a preferred embodiment, the present formulations are controlled release oral dosage forms, providing for controlled release, preferably sustained release, of any one of the active agents. Sustained release formulations provide for gradual release of one or more of the active agent(s), particularly the carbonic anhydrase inhibitor, from the dosage form to the patient's body over an extended time period, typically providing for a substantially constant blood level of the agent over a time period in the range of about 4 to about 12 hours, typically in the range of about 6 to about 10 hours. In a particularly preferred embodiment, there is a very gradual increase in blood level of the drug following oral administration of a combination dosage form containing the carbonic anhydrase inhibitor and the aldosterone antagonist, such that the peak blood level (generally about 50-200 μg/ml for topiramate, about 1-5 μg/ml for zonisamide, or about 10-35 μg/ml for acetazolamide), is not reached until at least 4-6 hours have elapsed, with the rate of increase of blood level drug approximately linear. In addition, in the preferred embodiment, there is an equally gradual decrease in blood level at the end of the sustained release period.

Generally, as will be appreciated by those of ordinary skill in the art, sustained release dosage forms are formulated by dispersing the active agents within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preferred sustained release dosage forms herein are composed of the acrylate and methacrylate copolymers available under the tradename “Eudragit” from Rohm Pharma (Germany). The Eudragit series E, L, S, RL, RS, and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

Each of the active agents may in addition be administered through the skin using conventional transdermal drug delivery systems, wherein the active agent or agents are contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the active agents may be formulated in a depot preparation for controlled release of the active agents, preferably sustained release over an extended time period. These sustained release dosage forms are generally administered by implantation (e.g., subcutaneously or intramuscularly or by intramuscular injection).

In combining the active agents herein, i.e., the carbonic anhydrase inhibitor with the aldosterone antagonist, the aldosterone antagonist will generally reduce the quantity of the carbonic anhydrase inhibitor needed to achieve a therapeutic effect when administered as a monotherapy, and, conversely, the carbonic anhydrase inhibitor will generally reduce the quantity of the aldosterone antagonist required.

As the method of the invention involves combination therapy, the active agents may be administered separately, at the same or at different times of day, or they be administered in a single pharmaceutical formulation. If a sympathomimetic amine is included, it is generally preferred that the amine compound be administered earlier in the day than the carbonic anhydrase inhibitor and the aldosterone antagonist. A single dosage form can contain the carbonic anhydrase inhibitor as well as the aldosterone antagonist and, optionally, the sympathomimetic amine, with the sympathomimetic amine preferably in immediate release form and the carbonic anhydrase inhibitor and the aldosterone antagonist in controlled release form. As an example, a combination dosage form of the invention for once-daily administration might contain (1) topiramate in the therapeutically effective amounts specified earlier herein, in controlled release (e.g., sustained release) form, (2) an aldosterone antagonist such as spironolactone, eplerenone, or potassium canrenoate, also in controlled release form, and (3) optionally, a sympathomimetic agent such as phentermine in immediate release form. Specific examples of such once-daily formulations include the following:
(1) 23 mg topiramate, 25 mg spironolactone;
(2) 23 mg topiramate, 50 mg spironolactone;
(3) 23 mg topiramate, 100 mg spironolactone;
(4) 23 mg topiramate, 25 mg eplerenone;
(5) 23 mg topiramate, 50 mg eplerenone;
(6) 23 mg topiramate, 100 mg eplerenone;
(7) 23 mg topiramate, 25 mg potassium canrenoate;
(8) 23 mg topiramate, 50 mg potassium canrenoate;
(9) 23 mg topiramate, 100 mg potassium canrenoate;
(10) 46 mg topiramate, 25 mg spironolactone;
(11) 46 mg topiramate, 50 mg spironolactone;
(12) 46 mg topiramate, 100 mg spironolactone;
(13) 46 mg topiramate, 25 mg eplerenone;
(14) 46 mg topiramate, 50 mg eplerenone;
(15) 46 mg topiramate, 100 mg eplerenone;
(16) 46 mg topiramate, 25 mg potassium canrenoate;
(17) 46 mg topiramate, 50 mg potassium canrenoate;
(18) 46 mg topiramate, 100 mg potassium canrenoate;
(19) 46 mg topiramate, 25 mg spironolactone;
(20) 92 mg topiramate, 50 mg spironolactone;
(21) 92 mg topiramate, 100 mg spironolactone;
(22) 92 mg topiramate, 25 mg eplerenone;
(23) 92 mg topiramate, 50 mg eplerenone;
(24) 92 mg topiramate, 100 mg eplerenone;
(25) 92 mg topiramate, 25 mg potassium canrenoate;
(26) 92 mg topiramate, 50 mg potassium canrenoate;
(27) 92 mg topiramate, 100 mg potassium canrenoate;
(28) 23 mg topiramate, 25 mg spironolactone, 7.5 mg phentermine;
(29) 23 mg topiramate, 50 mg spironolactone, 7.5 mg phentermine;
(30) 23 mg topiramate, 100 mg spironolactone, 7.5 mg phentermine;
(31) 23 mg topiramate, 25 mg eplerenone, 7.5 mg phentermine;
(32) 23 mg topiramate, 50 mg eplerenone, 7.5 mg phentermine;
(33) 23 mg topiramate, 100 mg eplerenone, 7.5 mg phentermine;
(34) 23 mg topiramate, 25 mg potassium canrenoate, 7.5 mg phentermine;
(35) 23 mg topiramate, 50 mg potassium canrenoate, 7.5 mg phentermine;
(36) 23 mg topiramate, 100 mg potassium canrenoate, 7.5 mg phentermine;
(37) 46 mg topiramate, 25 mg spironolactone, 7.5 mg phentermine;
(38) 46 mg topiramate, 50 mg spironolactone, 7.5 mg phentermine;
(39) 46 mg topiramate, 100 mg spironolactone, 7.5 mg phentermine;
(40) 46 mg topiramate, 25 mg eplerenone, 7.5 mg phentermine;
(41) 46 mg topiramate, 50 mg eplerenone, 7.5 mg phentermine;
(42) 46 mg topiramate, 100 mg eplerenone, 7.5 mg phentermine;
(43) 46 mg topiramate, 25 mg potassium canrenoate, 7.5 mg phentermine;
(44) 46 mg topiramate, 50 mg potassium canrenoate, 7.5 mg phentermine;
(45) 46 mg topiramate, 100 mg potassium canrenoate, 7.5 mg phentermine;
(46) 46 mg topiramate, 25 mg spironolactone, 7.5 mg phentermine;
(47) 92 mg topiramate, 50 mg spironolactone, 7.5 mg phentermine;
(48) 92 mg topiramate, 100 mg spironolactone, 7.5 mg phentermine;
(49) 92 mg topiramate, 25 mg eplerenone, 7.5 mg phentermine;
(50) 92 mg topiramate, 50 mg eplerenone, 7.5 mg phentermine;
(51) 92 mg topiramate, 100 mg eplerenone, 7.5 mg phentermine;
(52) 92 mg topiramate, 25 mg potassium canrenoate, 7.5 mg phentermine;
(53) 92 mg topiramate, 50 mg potassium canrenoate, 7.5 mg phentermine;
(54) 92 mg topiramate, 100 mg potassium canrenoate, 7.5 mg phentermine;
(55) 23 mg topiramate, 25 mg spironolactone, 150 mg bupropion;
(56) 23 mg topiramate, 50 mg spironolactone, 150 mg bupropion;
(57) 23 mg topiramate, 100 mg spironolactone, 150 mg bupropion;
(58) 23 mg topiramate, 25 mg eplerenone, 150 mg bupropion;
(59) 23 mg topiramate, 50 mg eplerenone, 150 mg bupropion;
(60) 23 mg topiramate, 100 mg eplerenone, 150 mg bupropion;
(61) 23 mg topiramate, 25 mg potassium canrenoate, 150 mg bupropion;
(62) 23 mg topiramate, 50 mg potassium canrenoate, 150 mg bupropion;
(63) 23 mg topiramate, 100 mg potassium canrenoate, 150 mg bupropion;
(64) 46 mg topiramate, 25 mg spironolactone, 150 mg bupropion;
(65) 46 mg topiramate, 50 mg spironolactone, 150 mg bupropion;
(66) 46 mg topiramate, 100 mg spironolactone, 150 mg bupropion;
(67) 46 mg topiramate, 25 mg eplerenone, 150 mg bupropion;
(68) 46 mg topiramate, 50 mg eplerenone, 150 mg bupropion;
(69) 46 mg topiramate, 100 mg eplerenone, 150 mg bupropion;
(70) 46 mg topiramate, 25 mg potassium canrenoate, 150 mg bupropion;
(71) 46 mg topiramate, 50 mg potassium canrenoate, 150 mg bupropion;
(72) 46 mg topiramate, 100 mg potassium canrenoate, 150 mg bupropion;
(73) 46 mg topiramate, 25 mg spironolactone, 150 mg bupropion;
(74) 92 mg topiramate, 50 mg spironolactone, 150 mg bupropion;
(75) 92 mg topiramate, 100 mg spironolactone, 150 mg bupropion;
(76) 92 mg topiramate, 25 mg eplerenone, 150 mg bupropion;
(77) 92 mg topiramate, 50 mg eplerenone, 150 mg bupropion;
(78) 92 mg topiramate, 100 mg eplerenone, 150 mg bupropion;
(79) 92 mg topiramate, 25 mg potassium canrenoate, 150 mg bupropion;
(80) 92 mg topiramate, 50 mg potassium canrenoate, 150 mg bupropion; and
(81) 92 mg topiramate, 100 mg potassium canrenoate, 150 mg bupropion.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description is intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

Formulations containing a carbonic anhydrase inhibitor and at least one additional active agent can be prepared as described herein. Evaluation of the combination therapy of the present methods and formulations in the treatment of OSAS can be carried out using known in vitro and in vivo techniques as described, for example, in Winslow et al. (2012) SLEEP 35 (11):1529-1539), the disclosure of which is incorporated by reference herein.

### Experimental

A double-blind, placebo-controlled, parallel-group study is conducted on randomized subjects fitting the following eligibility criteria: 30-65 years of age; body mass index (BMI) between 30 kg/m² and 40 kg/m²; a diagnosis of moderate to severe OSA syndrome, and an apnea-hypopnea index (AHI)≧15 at baseline (measured via overnight polysomnography [PSG]); and unwilling or unable to comply with positive air pressure (PAP) treatment (defined as >4 h/night, 70% of the time). Subjects are not eligible if they have a sleep disorder other than OSA syndrome, periodic limb movement arousal index >10, uncontrolled or poorly controlled blood pressure (systolic >160 mm Hg or diastolic >100 mm Hg), or the presence or history of unstable angina, heart failure, cardiac valvulopathy, myocardial infarction, potentially life-threatening cardiac arrhythmia, or clinically significant abnormality on electrocardiogram. Eligible subjects are randomized via a computer-generated table that assigns subjects to treatment arms (placebo versus topiramate/spironolactone combination) with equal probability. Both groups also receive standardized lifestyle modification counseling, e.g., using the LEARN program, a clinically proven behavioral weight loss and weight management program (K. Brownell, The LEARN Program for Weight Management,” Dallas: The Life Style Company, 2000).

Subjects are screened for eligibility, including overnight PSG to provide the baseline AHI value for each subject. Eligible subjects are then randomized 1:1 to receive either placebo or combination therapy comprising once-daily oral administration of 46 mg topiramate and 50 mg spironolactone. Both active agents are titrated at the outset, with an initial dosage of 23 mg topiramate and 25 mg spironolactone for the first four weeks, after which period dosage is increased to the maintenance dosages of 46 mg topiramate and 50 mg spironolactone. Subjects then receive an additional 24 weeks of treatment for a total treatment period of 28 weeks. Overnight assessments of OSA, including PSG, laboratory testing, and quality-of-life surveys are performed at baseline, Week 8, and Week 28.

The primary efficacy endpoint is the change in AHI between baseline, Week 8, and Week 28. An AHI score of 5-14 is considered mild, 15-29 is considered moderate, and ≧30 is considered severe. Secondary endpoints include changes in additional OSA parameters, such as respiratory disturbance index (RDI), apnea index, hypopnea index, desaturation index, mean overnight oxygen saturation, overnight minimum oxygen saturation, and arousal index (sleep quality defined as the number of arousals from REM and NREM sleep per hour). Subject-reported outcomes are also assessed, including the Pittsburgh Sleep Quality Index (PSQI), Epworth Daytime Sleepiness Scale (ESS), and 36-item Short-Form Health Survey (SF-36) scores. The PSQI is a subjective, self-administered questionnaire used to assess the quality and New Instrument for Psychiatric Practice and Research,” Psychiatry Res. 28:193-213). ESS is a subjective, self-administered, 8-question tool to assess excessive daytime sleepiness and is also used to differentiate between average and significant issues with sleepiness that require intervention; see M. W. Johns (1991), “A New Method for Measuring Daytime Sleepiness: The Epworth Sleepiness Scale,” Sleep 14:540-45; and M. W. Johns (1992), “Reliability and Factor Analysis of the Epworth Sleepiness Scale,” Sleep 15:376-81. The SF-36 questionnaire is a 36-item, self-administered health-related quality-of-life questionnaire designed to evaluate functional health and well-being (J. E. Ware et al. (1992), “The MOS 36-Item Short-Form Health Survey (SF-36): I. Conceptual Framework and Item Selection,” Med. Care 30:473-83).

Additional secondary endpoints to be noted include changes in cardiometabolic risk factors: blood pressure, heart rate, lipid profile (total cholesterol, low-density lipoprotein [LDL] cholesterol, high-density lipoprotein [HDL] cholesterol, and triglycerides), and glycemic variables (fasting insulin, fasting glucose, and insulin resistance based on homeostasis model of assessment—insulin resistance [HOMA-IR]). Percent weight loss and the percentage of subjects achieving ≧5% and 10% weight loss are also assessed as secondary endpoints. Safety endpoints include laboratory parameters, electrocardiogram, physical examination, and reports of adverse events. Adverse events are assessed throughout the study and coded using the Medical Dictionary for Regulatory Activities, version 10.1. Treatment-emergent adverse events (TEAE) are defined as adverse events that start on or after the first dose and up to 28 days after the last dose of study drug.

### Statistical Methods

Analysis of the primary efficacy variable, the change in AHI between baseline and Week 8, and between baseline and Week 28, with last observation carried forward (LOCF), is accomplished using an analysis of covariance (ANCOVA) model, with treatment groups as the main effect and baseline body weight as the covariate. The least-squares (LS) means and corresponding standard errors are calculated for the within-group AHI change for each treatment group. For the between-treatment group comparison, the difference in LS means, corresponding standard error, 95% confidence interval, and 1-sided P value can also be derived from this ANCOVA model. The normality assumption of the efficacy data is examined prior to fitting the ANCOVA models to ensure that normality is observed. The same statistical methodology described above for the analysis of the primary efficacy variable is performed for secondary variables. Analyses of percentage of categorical weight loss may be conducted using a logistic regression model with treatment as the fixed effect and baseline body weight as a covariate. For the between treatment group comparison, the estimated odds ratio, standard error, 2-sided 95% confidence interval, and 2-sided P value for treatment comparison are calculated.

### Results

Apnea/Hypopnea Index: The change from baseline in AHI, the primary endpoint, significantly favors the topiramate/spironolactone group versus placebo at both Week 8 and Week 28. The number of apnea-hypopnea events is reduced by an average of 20% to 80% relative to placebo, at both points of evaluation. In addition, at Week 28, significantly more subjects in the topiramate/spironolactone group with severe AHI (>45) at baseline are expected to achieve an AHI <5 by Week 28 than those receiving placebo.

Weight Loss and Correlation between Improvements in Weight and Apnea/Hypopnea Index: At Week 8, absolute mean weight loss is greater in the topiramate/spironolactone group than in the placebo group, with the trend continuing at Week 28.

Overnight Polysomnography Endpoints: Analysis of the secondary overnight PSG endpoints will reveal changes between baseline and Week 28 for additional OSA parameters. Statistically significant changes are expected to favor topiramate/spironolactone therapy versus placebo for RDI, mean overnight oxygen saturation, and hypopnea index. Both treatment groups have a similar number of arousals and arousal index score at baseline. By Week 8, both groups may experience a reduction in the arousal index, with a greater decrease observed, as expected, in the topiramate/spironolactone group. By Week 28, the difference between the groups increases substantally.

Sleep Quality: Secondary analyses of sleep-quality indices are expected to show a significant difference between topiramate/spironolactone therapy and placebo in the mean change in PSQI at Week 28. ESS evaluation and SF-36 quality-of-life parameters at Weeks 8 and 28 are expected to favor topiramate/spironolactone therapy relative to placebo as well.

The findings of the randomized controlled trial demonstrate significant improvements in OSA using 46 mg topiramate and 50 mg spironolactone combined with lifestyle interventions at 28 weeks. The study shows a correlation between improvements in AHI and weight loss and suggests that topiramate/spironolactone therapy combined with lifestyle interventions may be useful as a primary treatment for OSA in patients who are unable or unwilling to use CPAP therapy.

The above protocol may be repeated with any or all of the active agent combinations provided earlier herein (e.g, 23 mg, 46 mg, 92 mg topiramate with 25 mg, 50 mg, 100 mg spironolactone; 23 mg, 46 mg, 92 mg topiramate with 25 mg, 50 mg, 100 mg eplerenone; 23 mg, 46 mg, 92 mg topiramate with 25 mg, 50 mg, 100 mg potassium canrenoate; and the aforementioned combinations with 7.5 phentermine or 150 mg bupropion). Substantially the same results are expected.

We claim:

1. A method for treating sleep apnea in a patient, comprising co-administering to the patient a therapeutically effective amount of a carbonic anhydrase inhibitor and a therapeutically effective amount of an aldosterone antagonist.

2. The method of claim 1, wherein the carbonic anhydrase inhibitor is an anti-epileptic agent.

3. The method of claim 2, wherein the carbonic anhydrase inhibitor is a sulfamate compound.

4. The method of claim 2, wherein the carbonic anhydrase inhibitor is a sulfonamide compound.

5. The method of claim 1, wherein the carbonic anhydrase inhibitor is selected from acetazolamide, brinzolamide, diclofenamide, dichlorphenamide, dorzolamide, furosemide, imidazole, methazolamide, phenylalanine, topiramate, zonisamide, celecoxib, valdecoxib, rofecoxib, and etoricoxib.

6. The method of claim 5, wherein the carbonic anhydrase inhibitor is acetazolamide.

7. The method of claim 5, wherein the carbonic anhydrase inhibitor is zonisamide.

8. The method of claim 5, wherein the carbonic anhydrase inhibitor is topiramate.

9. The method of claim 1, wherein the aldosterone antagonist has the molecular structure of Formula (I) 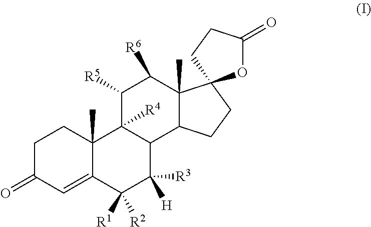
wherein:
(a) R¹ and R² are independently selected from H and OH, and R³ is selected from —COOR⁷ and —S(CO)R⁸ in which R⁷ and R⁸ are lower alkyl, or R¹ is H and R² and R³ taken together form a double bond or a cyclopropyl ring;
(b) R⁴ and R⁵ are independently selected from H and OH, or R⁴ and R⁵ taken together form an epoxide ring; and
(c) R⁶ is selected from H and OH,
or is a pharmaceutically acceptable basic addition salt thereof.

10. The method of claim 9, wherein R¹ is H, R² and R³ taken together form a double bond, and R⁶ is H, such that the compound has the structure of Formula (III)

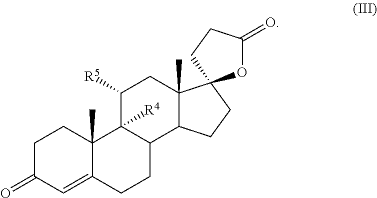

11. The method of claim 9, wherein R¹, R², and R⁶ are H, such that the compound has the structure of Formula (IV):

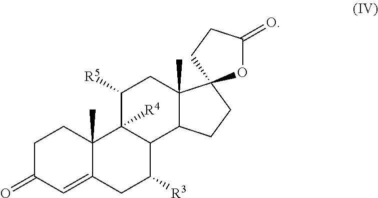

12. The method of claim 11, wherein R³ is selected from —COOCH₃ and —SOCH₃.

13. The method of claim 1, wherein the aldosterone antagonist is selected from spironolactone, canrenone, eplerenone, mexrenone, prorenone, and pharmaceutically acceptable basic addition salts thereof.

14. The method of claim 13, wherein the aldosterone antagonist is selected from spironolactone, eplerenone, and potassium canrenoate.

15. The method of claim 1, further including co-administering a therapeutically effective amount of a sympathomimetic amine.

16. The method of claim 9, wherein the sympathomimetic amine is selected from amphetamine, benzphetamine, bupropion, chlorphentermine, colterol, diethylpropion, dopamine, dobutamine, ephedrine, epinephrine, epinine, ethylnorepinephrine, fenfluramine, fenoldapam, hydroxyamphetamine, ibopamine, isoetharine, isoproterenol, mephentermine, metaproterenol, metaraminol, methoxamine, methoxyphenamine, midodrine, norepinephrine, phendimetrazine, phenmetrazine, phentermine, phenylephrine, phenylethylamine, phenylpropanolamine, prenalterol, propylhexedrine, protokylol, ritodrine, terbutaline, tuaminoheptane, tyramine, pharmaceutically acceptable salts thereof, and combinations of any of the foregoing.

17. The method of claim 16, wherein the sympathomimetic amine is phentermine.

18. The method of claim 1, wherein the carbonic anhydrase inhibitor and the aldosterone antagonist are administered simultaneously.

19. The method of claim 18, wherein the carbonic anhydrase inhibitor and the aldosterone antagonist are administered in a single pharmaceutical formulation that further includes a pharmaceutically acceptable excipient.

20. The method of claim 19, wherein the pharmaceutical formulation comprises the carbonic anhydrase inhibitor in controlled release form.

21. The method of claim 20, wherein the pharmaceutical formulation further comprises the aldosterone antagonist in controlled release form.

22. The method of claim 19, wherein the pharmaceutical formulation further comprises a sympathomimetic amine in immediate release form.

23. The method of claim 19, wherein the pharmaceutical formulation comprises a unit dosage form for once-daily administration.

24. The method of claim 1, wherein the carbonic anhydrase inhibitor and the additional active agent are administered orally.

25. A method for treating obstructive sleep apnea syndrome in a patient, comprising orally administering to the patient:
a therapeutically effective amount of topiramate;
a therapeutically effective amount of an aldosterone antagonist selected from spironolactone, eplerenone, and potassium canrenoate; and, optionally,
a therapeutically effective amount of phentermine.

26. A pharmaceutical formulation comprising a therapeutically effective amount of a carbonic anhydrase inhibitor and a therapeutically effective amount of an aldosterone antagonist.

27. A pharmaceutical formulation comprising topiramate and an aldosterone antagonist selected from spironolactone, canrenone, eplerenone, mexrenone, prorenone, and pharmaceutically acceptable basic addition salts thereof.

28. The pharmaceutical formulation of claim 27, wherein the aldosterone antagonist is selected from spironolactone, eplerenone, and potassium canrenoate.

29. A packaged pharmaceutical preparation comprising a container housing at least one dosage form each comprising a therapeutically effective amount of a carbonic anhydrase inhibitor and a therapeutically effective amount of an aldosterone antagonist.
